(19) United States
(12) Patent Application Publication
(10) Pub. No.: US 2025/0072525 A1
(43) Pub. Date: Mar. 6, 2025

(54) DRESS WITH A CLOTH EXTENDER

(71) Applicant: Vashi T. Balani, Mataki (PH)

(72) Inventor: Vashi T. Balani, Mataki (PH)

(21) Appl. No.: 18/643,956

(22) Filed: Apr. 23, 2024

(30) Foreign Application Priority Data
Sep. 4, 2023 (PH) 12023050489

(51) Int. Cl. A41B 13/08

(52) U.S. Cl. CPC A41B 13/08; A41B 2300/324

(57) ABSTRACT

The present invention generally relates to a wearable dress with an attachment, more particularly to be known as a dress with a cloth extender. It comprises a dress with a cloth extender that has piping. On the bottom portion of the fabric is a lower cloth lining provided with a plurality of male fastening means. And In the top portion of the fabric is the upper cloth lining with a plurality of female fastening means, which is a female snap button that is attached to said upper cloth lining. The snap buttons are needed to attach the cloth extender to the button of the cloth. The dress with an extender will provide additional expansion of the lower portion of the clothes, making them more comfortable to wear. The cloth extender is made mostly from the same fabric and matches in color with the dress.

### TECHNICAL FIELD

The present invention generally relates to a wearable dress with an attachment. More particularly, a dress with a cloth extender configured as an extension or adjustment to a dress that will make the old or outgrown cloth dress to be usable again.

### BACKGROUND OF THE INVENTION

A variety of cloth or dresses exists in the prior art, whose primary objective is to provide a means for protecting the human body. From baby up to the stage of adulthood, clothing size is typically based on age. Also, personal fashion is a social-cultural consumerist practice that encodes in people's fashion the representation of many social features and depicts a system characterized by differences in social class, richness, gender, or ethnicity.

Clothes or dresses are sometimes sewn with a snap at the crotch. These snaps help fit the fabric clothing perfectly to the body and help facilitate changing underwear or diapers as it opens and closes in the crotch area. However, given that humans grow bigger and taller rapidly, cloth extenders are beneficial for using dresses for long periods. By making the dress last longer and reusable, helps to reduce carbon footprint. Different companies have different ways of stitching or attaching the snaps; for example, the use of different diameters of the snaps used in the dress and the extender can cause a mismatch. Also, using different quality snaps has snaps made of plastic; some are nickel plated, and some are metal plated, etc. Also contributes to the action of mismatching the extenders with the dress. Even a slight change in the position of the attachment of the snaps, either in the dress or the extender, would cause the failure of the attachment of the extender to the dress. Thus, snaps mismatches when bought from various businesses. Therefore, a invention for clothes or dresses with cloth extenders with a standard arrangement of Snap-on buttons would eliminate the mismatch problem.

Since that human grows fast, the garment soon comes to the stage of being discarded. However, using clothes with cloth extenders will bring new life to the garment With a cloth extender, the end user will be more comfortable, and with the extender attached to the clothing, the muscles will lose due to the release of pressure from the outgrown clothing, making the user more comfortable wearing the clothing or dress.

The cloths used in the invention are stitched using either knit or woven fabrics, and the fabric is cotton or any kind. The extenders are made from the same fabrics and can be matched in color and the snaps' quality.

### SUMMARY OF THE INVENTION

The present invention generally relates to a wearable dress with an attachment. More particularly, a dress with a cloth extender configured as an extension or adjustment to a dress that will make the old or outgrown cloth dress to be usable again.

The present invention generally relates to a wearable dress with an attachment, more particularly to be known as a dress with a cloth extender. It comprises a dress with a cloth extender that has piping. On the bottom portion of the fabric is a lower cloth lining provided with a plurality of male fastening means. And In the top portion of the fabric is the upper cloth lining with a plurality of female fastening means, which is a female snap button that is attached to said upper cloth lining. The snap buttons are needed to attach the cloth extender to the button of the cloth. The dress with an extender will provide additional expansion of the lower portion of the clothes, making them more comfortable to wear. The cloth extender is made mostly from the same fabric and matches in color with the dress.

### BRIEF DESCRIPTION OF THE DRAWINGS

The present invention will be better understood by reference to the accompanying drawings, which:

FIG. 1 is a front view of a dress and a cloth extender.

FIG. 2 is a perspective view of the cloth extender.

FIG. 3 is a view of the present embodiment of a dress with a cloth extender.

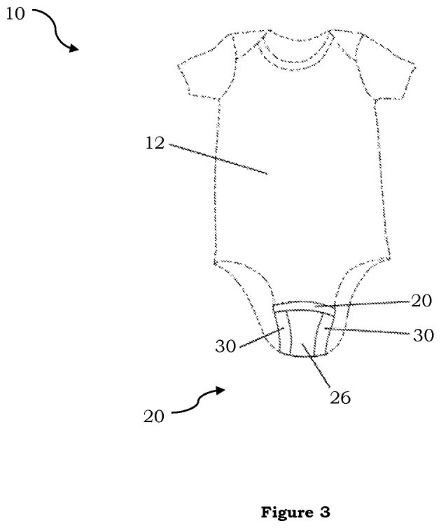

### DETAILED DESCRIPTION

Before describing the present invention in detail, it is to be understood that the phraseologies and terminologies used herein are for the purposes of description and should not be regarded as limiting.

It will be understood that although the terms first, second, third, etc., may be used to describe various elements, these elements should not be limited by these terms. These terms may be used to distinguish one element from another. For example, a first element may be termed a first element without departing from the scope of the present invention. As used herein, the term “and/or” may include all combinations of one or more of the listed items.

Referring now to the drawings, wherein like reference numerals, designate the components or elements throughout the ensuing enabling description, the present invention provides a dress with a cloth extender designated as 10.

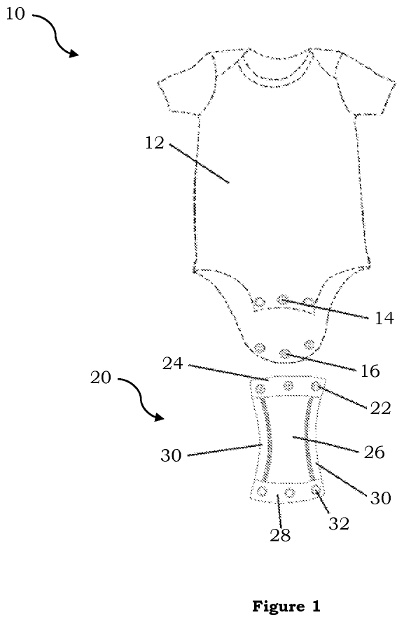

In reference to FIG. 1, the dress with a cloth extender 10 comprises a dress 12, a plurality of first set female fastening means 14 attached on the lower front side of the dress 12, a plurality of first set male fastening means 16 attached on the lower rear side of the dress 12, and a cloth extender 20.

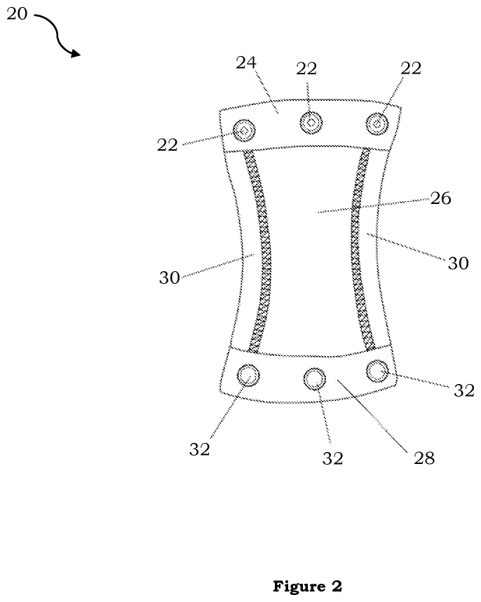

The cloth extender 20, as shown in FIG. 2, further comprises a plurality of second-set male fasting means 22 being fixedly attached to a front cloth lining 24, a cloth 26 fixedly sewn in between the front cloth lining 24 and a rear cloth lining 28, a piping cloth 30 sewn on the side of the cloth 26 and wherein said piping cloth 30 is provided to cover the side cutting edges of the cloth extender 20 and preventing the cloth 26 to be stretched out when attached to the dress 12, and a plurality of second set female fasting means 32 attached to the rear cloth lining 28. The cloth 12 is preferably cotton or another kind of fabric used for clothes being breathable and absorbent fabric.

When used, the plurality of second-set female fasting means 32 are snapped and attached to the plurality of first-set male fastening means 16, while the plurality of first-set female fasting means 14 are snapped and attached to the plurality of second-set male fastening means 22. The dress with a cloth extender 10 has the same kind of color combination and is made mostly from the same fabric and matched color. Furthermore, said dress with a cloth extender 10 is characterized by said cloth extender 20 being configured as an extension or adjustment to the dress 12 that will make the old or outgrown cloth dress to be usable again.

Lastly, the plurality of first set of female fastening means 14, the plurality of second set of female fastening means 32, the plurality of first set of male fastening means 16, and the plurality of second set of male fastening 22 used snap buttons for the great quality, durability, and strength for rugged use.

Additional advantages and modifications of the present invention will readily occur to those skilled in the art in view of these teachings. The present invention, in its broader aspects, is not limited to the specific details, representative contrivances, and illustrative examples shown and described herein. Accordingly, various modifications may be made without departing from the spirit and scope of the general concept as defined in the appended claims and its equivalents.

The embodiments of the invention in which an exclusive property or privilege is claimed are defined as follows:

1. A dress with a cloth extender (10) comprising:
a dress (12),
a plurality first set female fastening means (14) attached to the lower front side of the dress (10),
a plurality of the first set of male fastening means (16) attached on the lower rear side of the dress (12),
a cloth extender (20) being attachable to the dress (12) and wherein said cloth extender (20) further comprises:
a plurality of second-set male fasting means (22) being fixedly attached to a front cloth lining (24),
a cloth (26) fixedly sewn in between the front cloth lining (24) and a rear cloth lining (28),
a piping cloth (30) sewn on the side of the cloth (26),
a plurality of second-set female fasting means (32) attached to the rear cloth lining (28),
characterized of said cloth extender (20) when attached to the dress (12) through the plurality of second-set female fasting means (32) being snapped and attached to the plurality of first-set male fastening means (16) and the plurality of first-set female fasting means (14) to the plurality of second-set male fastening means (22) provides an extension and adjustment means to the dress (12) enabling said dress 12 to be usable again.

2. The dress with a cloth extender (10), according to claim 1, wherein said plurality of first set of female fastening means (14), the plurality of second set of female fastening means (32), the plurality of first set of male fastening means (16), and the plurality of second set of male fastening (22) are snap buttons.

3. The dress with a cloth extender (10), according to claim 1, wherein said dress (12) and cloth (26) have the same kind of fabric.

4. The dress with a cloth extender (10), according to claim 1, wherein said dress (12) and said cloth (26) have the same kind of color combination.

5. The dress with a cloth extender (10), according to claim 1, wherein said piping cloth (30) is provided to cover the side cutting edges of the cloth extender (20) and preventing the cloth (26) from being stretched out when attached to the dress (12).
